(19) United States
(12) Patent Application Publication
(10) Pub. No.: US 2010/0093673 A1
(43) Pub. Date: Apr. 15, 2010

(54) PAIN RELIEVING PATCH

(76) Inventor: Bryan T. ORONSKY, Los Altos Hills, CA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/501,924

(22) Filed: Jul. 13, 2009

Related U.S. Application Data
(60) Provisional application No. 61/134,890, filed on Jul. 14, 2008.

(51) Int. Cl. A61K 31/60; A61K 47/22; A61K 31/195; A61K 31/19; A61K 31/415; A61K 31/54; A61K 31/445; A61K 31/40; A61P 29/00

(52) U.S. Cl. 514/159; 514/788; 514/567; 514/568; 514/403; 514/227.5; 514/315; 514/408

(57) ABSTRACT

Described here are patches for the treatment of pain. The patches include a carrier material, which itself, in the absence of an active agent, is capable of relieving pain. Methods of treating pain using the patches are also described.

### CROSS-REFERENCE TO RELATED APPLICATIONS

This application claims priority to U.S. Provisional Patent Application Ser. No. 61/134,890 filed on Jul. 14, 2008, which is hereby incorporated by reference in its entirety.

### FIELD

Described here are analgesic patches. The analgesic patches include a carrier material, and in the absence of an active agent, are capable of treating pain. Methods for treating various type of pain using the analgesic patches are also described.

### BACKGROUND

Nonsteroidal anti-inflammatory drugs (NSAIDs) have been widely used in the treatment of pain. In brief, NSAIDs decrease inflammation (and thus, pain) by inhibiting the biosynthesis of prostaglandins, which are responsible for inflammatory vasodilation and recruitment of other inflammatory cells. Specifically, NSAIDs prevent the conversion of arachadonic acid in cellular membranes to prostaglandins by inhibiting the activity of the enzyme cyclooxygenase.

Oral NSAIDs are typically used for pain, and can be provided in prescription strength or in lower doses over the counter. However, these oral medications are associated with serious gastrointestinal side-effects. For example, gastric upset, gastrointestinal bleeding, and gastric ulcers may develop with chronic use or high doses of oral NSAIDs. Chronic use and high doses are oftentimes necessary when treating conditions such as arthritis, cervical/lumbar spondylosis, and fibromyalgia.

Accordingly, topical preparations for treating pain would be useful. Transdermal preparations with simple administration and precise dosing would also be desirable. In particular, patches capable of treating pain would be desirable.

### SUMMARY

Described here are patches and methods for treating pain. The analgesic patches include a carrier material without an active agent. The carrier material may be a polymer, e.g., a hydrophilic polymer, hydrophobic polymer, pressure-sensitive adhesive polymer, and the like. In one variation, the carrier material includes hydroxyethylpyrrolidine. In another variation, the carrier material includes hydroxyethylpiperidine. Other components such as penetration enhancers, thickeners, preservatives, etc., may also be incorporated into the carrier material. The analgesic patches may be applied to treat pain of any etiology. They may be applied for any time period, and reapplied if desired.

### DETAILED DESCRIPTION

Surprisingly, it has been discovered that in the absence of an active agent, the inactive materials themselves, e.g., the carrier material, of a transdermal patch can relieve pain. In one variation, the Flector® patch (Institut Biochemique SA, Lugano, Switzerland) relieves pain when it is applied without diclofenac epolamine, which is normally included as the active nonsteroidal anti-inflammatory agent. As used herein, the term “analgesic patch” refers to a transdermal patch without an active agent. Thus, the Flector® patch lacking diclofenac epolamine would be an analgesic patch.

I. Analgesic Patches

The analgesic patches described here include a carrier material without an active agent. The analgesic patches may be of any dimension and geometry. For example, they may be of any length, width, or thickness. They may be formed as squares, rectangles, ovals, etc. Dimensions and shape of the analgesic patches may depend on such factors as the location to which the patch is being applied and amount of carrier material desired for application to the skin. It will be understood that the term “skin” includes any skin layer and mucosa. The analgesic patches may be configured to allow shaping of the carrier material by cutting. In other variations, the analgesic patches are provided as preformed shapes.

The analgesic patches may have a width of at least about 3.0 cm, at least about 5.0 cm, or at least about 10 cm. They may also have a length of at least about 5.0 cm, at least about 10 cm, or at least about 14 cm. The analgesic patches may include a single layer or multiple layers.

The carrier material generally contains a polymer. Any polymer may be used. For example, hydrophobic polymers, hydrophilic polymers, and/or pressure-sensitive adhesive (PSA) polymers may be employed. Suitable hydrophilic polymers include, but are not limited to, hydroxyethylpyrrolidine, hydroxyethylpiperidine, and the like. Hydrophobic polymers that may be used include, but are not limited to, polysiloxanes, polyacrylates, and polyisobutylene. Suitable PSA polymers that may be included in the analgesic patches include, without limitation, polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene).

The carrier material of the analgesic patches may include various other components. For example, thickening agents, humectants, fillers, preservatives, cross-linking agents, penetration enhancers, and others commonly employed in the pharmaceutical field may be incorporated. For instance, thickening agents such as polyacrylic acid, sodium polyacrylate, carboxymethylcellulose sodium (CMC Na), polyvinyl alcohol, polyvinylpyrrolidone, gelatin, and others may be used. The amount used may be between about 3.0 to about 30% by weight, or between about 5.0 to about 20% by weight.

Exemplary humectants for use with the analgesic patches include glycerol, propylene glycol, polyethylene glycol, 1,3-butanediol, and D-sorbitol solution. The amount used may be within a range of about 5.0 to about 70% by weight, preferably about 10% to about 60% by weight. Examples of fillers include kaolin and bentonite. Examples of preservatives are paroxybenzoic acid esters and sorbic acid. Examples of cross-linking agents are aluminum compounds and calcium compounds. The amount used may be within a range of about 0.01% to about 3.0% by weight. 1,3-butanediol (1,3-butylene glycol) is an exemplary penetration enhancer that may be included in the analgesic patches described here, in the range of about 10% to about 30%, or more. Other types of penetration enhancers, thickening agents, humectants, fillers, preservatives, and cross-linking agents may be employed, and other amounts used to make up for the lack of an active agent within the carrier.

Other irritation-mitigating additives may also be incorporated into the analgesic patches to minimize or eliminate the possibility of skin irritation. Suitable irritation-mitigating additives include, for example, alpha-tocopherol; monoamine oxidase inhibitors, e.g., phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine.

In one variation, an analgesic patch having the following components is used to treat pain: 1,3-butylene glycol, dihydroxyalumninum aminoacetate, disodium edetate, D-sorbitol, fragrance (Dalin PH), gelatin, kaolin, methylparaben, polysorbate 80, povidone, propylene glycol, propylparaben, sodium carboxymethylcellulose, sodium polyacrylate, tartaric acid, titanium dioxide, and purified water. Here the components may be included in the following amounts by weight: about 10% to about 30% 1,3-butylene glycol, about 4% to about 8% sodium polyacrylate, about 3% to about 6% sodium carboxymethylcellulose, about 2% to about 4% gelatin, about 2% to about 4% povidone, about 20% to about 40% D-sorbitol, about 5% to about 10% kaolin, about 0.5% to about 1% titanium dioxide, about 0.8% to about 1.5% dihydroxyalumninum aminoacetate, about 0.3% to about 0.5% tartaric acid, about 0.1% to about 0.5% methylparaben, about 0.05% to about 0.1% propylparaben, and the rest, water. The aforementioned amounts are the amounts of the components provided in the commercially available Flector® patch.

A backing layer may also be included with the analgesic patches, e.g., to provide structural support to the patch, and in certain variations, occlusivity. The backing may be comprised of a flexible elastomeric material that serves as a protective covering to prevent transmission of substances through the upper surface of the patch, and may impart a degree of occlusivity to the patch, such that the area of the body surface covered by the patch becomes hydrated during use. The material used for the backing layer may permit the patch to follow the contours of the skin and be worn comfortably on areas such as joints or other points of flexure that are normally subjected to mechanical strain, with little or no likelihood of the patch disengaging from the skin due to differences in the flexibility or resiliency of the skin and the patch. The materials used as the backing layer may be either occlusive or permeable, as noted above, and may be made from synthetic polymers (e.g., polyester, polyethylene, polypropylene, polyurethane, polyvinyl chloride, and polyether amide), natural polymers (e.g., cellulosic materials), or macroporous woven and nonwoven materials.

During storage and prior to use, the analgesic patches may include a release liner. Immediately prior to use, this layer is typically removed so that the patches may be affixed to the skin. The release liner may be prepared as a disposable element that serves only to protect the patch prior to application. The release liner may be formed from a material impermeable to the polymer of the carrier material and other substances contained therein, and which is easily stripped from the analgesic patch prior to use.

The analgesic patch may be made by dissolving or dispersing a part of the above components in water and kneading with other components. A pH adjusted may then be added if desired. In the course of preparation, the addition order of a pH adjuster is not particularly limited. Thus, when an acidic thickening agent is added, the pH may be adjusted by addition of an alkaline pH adjuster, and vice versa when an alkaline thickening agent is used. In one variation, the analgesic patches are fabricated using conventional coating and laminating techniques known in the art. For example, adhesive matrix systems can be prepared by casting a fluid admixture of adhesive, active agent, and carrier onto the backing layer, followed by lamination of the release liner. In another variation, the adhesive mixture may be cast onto the release liner, followed by lamination of the backing layer. The patches may also be fabricated by such processes as solvent evaporation, film casting, melt extrusion, thin film lamination, die cutting, and the like.

II. Methods for Analgesic Patches

The analgesic patches described here may be applied to any painful area or any area adjacent thereto. The analgesic patches may be applied to obtain total or partial relief of pain, or to prevent pain. The analgesic patches may also be applied for any time period. For example, they may be applied over one hour or less, over several hours (e.g., two to four hours, or two to six hours), over a period of one day to several days, over a period of one week to several weeks, or over a period of one month to several months or more. The analgesic patches may be reapplied until the desired amount of pain relief has been obtained or otherwise.

The analgesic patches may be used to treat any type of pain. The pain may be acute or chronic in nature. Types of pain where the analgesic patches may be helpful, include, but are not limited to, arthritic pain, joint pain, muscle pain, nerve pain, pain due to sprains or bruises, or pain due to inflammation. In view of this, the analgesic patches may be helpful in treating pain associated with conditions such as rheumatoid arthritis, lupus, Reiter's syndrome, fibromyalgia, diabetic neuropathy, etc.

III. Other Applications

As previously mentioned, the inactive ingredients of a transdermal patch, e.g., the Flector® patch, has been surprisingly found to relieve pain when it is applied without its active agent. Here the carrier material itself is capable of relieving pain when the patch is applied to the skin at or adjacent to a painful area. It has also been surprisingly found that a NSAID-containing patch such as the Flector® patch may be useful in suppressing appetite. As used herein, the term “adhesive patch” refers to a patch including an NSAID that is used in suppressing appetite.

The adhesive patches described here may include the same carrier materials (polymers, penetration enhancers, thickening agents, humectants, fillers, preservatives, and cross-linking agents) as mentioned above for the analgesic patches. A backing and release liner may also be provided as described above. Unlike the analgesic patches, the adhesive patches contain one or more NSAIDs.

Exemplary NSAIDs that may be included in the adhesive patches include, but are not limited to, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, and combinations thereof.

Suitable aminocarboxylic acid derivatives that may be used include, without limitation, enfenamic acid, etofenamate, flufenamic acid, isonixin, meclofenamic acid, mefenamic acid, niflumic acid, talniflumate, terofenamate, tolfenamic acid, and combinations, salts, and derivatives thereof.

Suitable arylacetic acid derivatives that may be used include, without limitation, aceclofenac, acemetacin, alclofenac, amfenac, amtolmetin guacil, bromfenac, bufexamac, diclofenac, etodolac, felbinac, fenclozic acid, fentiazac, glucametacin, ibufenac, indomethacin, isoxepac, lonazolac, metiazinic acid, mofezolac, nepafenac, oxametacine, proglumetacin, sulindac, tiaramide, tolmetin, tropesin, zomepirac, and combinations, salts, and derivatives thereof. In one variation, diclofenac epolamine is used.

Arylbutyric acid derivatives that may be employed in the adhesive patches described here include, but are not limited to bumadizon, butibufen, butixirate, fenbufen, and combinations, salts, and derivatives thereof.

Arylcarboxylic acids that may be used include, but are not limited to, ketorolac, tinoridine, or combinations, salts, or derivatives thereof.

Arylpropionic acid derivatives that may be employed include, without limitation, alminoprofen, bermoprofen, carprofen, fenoprofen, flunoxaprofen, flurbiprofen, ibuprofen, ibuproxam, ketoprofen, loxoprofen, naproxen, oxaprozin, piketoprofen, pranoprofen, suprofen, tiaprofenic acidximoprofen, zaltoprofen, and combinations, salts, and derivatives thereof.

Pyrazoles that may be used include difenamizole, epirizole, or combinations, salts, or derivatives thereof. Suitable pyrazolones that may be included in the adhesive patches described here include, but are not limited to, apazone, feprazone, mofebutazone, morazone, oxyphenbutazone, phenylbutazone, pipebuzone, propyphenazone, ramifenazone, suxibuzone, and combinations and derivatives thereof.

Exemplary salicylic acid derivatives include, but are not limited to, acetaminosalol, aspirin, balsalazide, benorylate, calcium acetylsalicylate, diflunisal, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salicylamide O-acetic acid, salicylsulfuric acid, salsalate, sodium salicylate, sulfasalazine, and combinations and derivatives thereof.

Exemplary thiazinecarboxamides include, but are not limited to, ampiroxicam, lornoxicam, meloxicam, piroxicam, tenoxicam, and combinations and derivatives thereof.

Other NSAIDs that may be employed in the adhesive patches described here include, without limitation, ε-acetamidocaproic acid, S-adenosylmethionine, ajulemic acid, 3-amino-4-hydroxybutyric acid, bendazac, benzydamine, α-bisabolol, bucolome, celecoxib, difenpiramide, ditazol, emorfazone, etoricoxib, fepradinol, guaiazulene, lexipafant, licofelone, lumiracoxib, nabumetone, nimesulide, oxaceprol, parecoxib, perisoxal, proquazone, rofecoxib, tenidap, valdecoxib, and combinations and derivatives thereof.

The adhesive patches described here may be applied to any part of the abdomen. They may be applied to completely eliminate hunger, to obtain partial suppression of appetite, to prevent hunger pains, or to prevent food cravings. The adhesive patches may also be applied for any time period. For example, they may be applied for one hour or less, over several hours (e.g., two to four or six hours), over a period of one day to several days, over a period of one week to several weeks, or over a period of one month to several months or more. The adhesive patches may be reapplied until the desired amount of appetite suppression has been obtained. The adhesive patches may also be used in combination with other commercially available oral appetite suppressants.

1. An analgesic patch comprising a carrier material, wherein the carrier material itself, in the absence of an active agent, is capable of treating pain.

2. The analgesic patch of claim 1 wherein the carrier material comprises hydroxyethylpyrrolidone or hydroxyethylpiperidine.

3. The analgesic patch of claim 1 wherein the carrier material further comprises a penetration enhancer, a pH adjuster, a thickening agent, a humectant, a filler, a preservative, a crosslinking agent, or combinations thereof.

4. The analgesic patch of claim 1 wherein the carrier material further comprises a penetration enhancer.

5. The analgesic patch of claim 1 wherein the penetration enhancer comprises 1,3-butylene glycol.

6. A method for treating pain comprising applying the analgesic patch of claim 1 to the skin at or adjacent to a painful area.

7. The method of claim 6 wherein the analgesic patch is applied to treat acute pain, chronic pain, arthritic pain, joint pain, muscle pain, nerve pain, sprains, bruises, or pain due to inflammation.

8. The method of claim 6 wherein the analgesic patch is reapplied to the skin.

9. A method for suppressing appetite comprising applying an adhesive patch to the abdomen, wherein the adhesive patch comprises a nonsteroidal anti-inflammatory agent and a carrier material.

10. The method of claim 9 wherein the nonsteroidal anti-inflammatory agent is an aminoarylcarboxylic acid derivative, an arylacetic acid derivative, an arylbutyric acid derivative, an arylcarboxylic acid, an arylpropionic acid derivative, a pyrazole, a pyrazolone, a salicylic acid derivative, a thiazinecarboxamide.

11. The method of claim 10 wherein the nonsteroidal anti-inflammatory agent comprises an arylacetic acid derivative.

12. The method of claim 11 wherein the arylacetic acid derivative is selected from the group consisting of aceclofenac, acemetacin, alclofenac, amfenac, amtolmetin guacil, bromfenac, bufexamac, diclofenac, etodolac, felbinac, fenclozic acid, fentiazac, glucametacin, ibufenac, indomethacin, isoxepac, lonazolac, metiazinic acid, mofezolac, nepafenac, oxametacine, proglumetacin, sulindac, tiaramide, tolmetin, tropesin, zomepirac, and combinations, salts, and derivatives thereof.

13. The method of claim 1 wherein the nonsteroidal anti-inflammatory agent comprises diclofenac epolamine.

14. The method of claim 1 wherein the carrier material comprises hydroxyethylpyrrlidone or hydroxyethypiperidine.

15. The method of claim 1 wherein the adhesive patch is reapplied to the abdomen.

16. A Flector® patch without diclofenac epolamine, wherein the patch is capable of treating pain.

17. A method for treating pain comprising applying the Flector® patch of claim 16 to the skin at or adjacent to a painful area.

18. A method for suppressing appetite comprising applying the Flector® patch having diclofenac epolamine to the abdomen.
